(19) United States
(12) Patent Application Publication
(10) Pub. No.: US 2025/0072795 A1
(43) Pub. Date: Mar. 6, 2025

(54) Flashing Indicator of Swimmer's Health

(71) Applicant: Government of the United States, as represented by the Secretary of the Air Force, Wright-Patterson AFB, OH (US)

(72) Inventors: Gregory M BURNETT, Dayton, OH (US); Peter J VOLAND, Beavercreek, OH (US); David P SARDO, Dayton, OH (US); Matthew M STECHSCHULTE, Beavercreek, OH (US)

(21) Appl. No.: 18/598,630

(22) Filed: Mar. 7, 2024

Related U.S. Application Data
(62) Division of application No. 16/386,835, filed on Apr. 17, 2019, now abandoned.
Application No. 16/386,835 is a continuation-in-part of application No. 14/755,020, filed on Jun. 30, 2015, now Pat. No. 10,413,225.
(60) Provisional application No. 62/658,651, filed on Apr. 17, 2018.

(51) Int. Cl. A61B 5/1455; A61B 5/00; A61B 5/0205; A61B 5/024

(52) U.S. Cl. CPC A61B 5/14551; A61B 5/02055; A61B 5/02427; A61B 5/14552; A61B 5/681; A61B 5/6824; A61B 5/6826; A61B 5/0059; A61B 5/02438; A61B 5/6803; A61B 2503/10

(57) ABSTRACT

A waterproof pulse oximeter sensor assembly configured to monitor vitals of a swimmer including a pulse oximeter sensor configured to evaluate conditions in a finger or a wrist of a swimmer and generate a pulse-ox signal in response thereto. A waterproof electronics compartment includes electronics configured to electrically receive the pulse-ox signal from the pulse oximeter sensor, and determine a heartbeat rate and/or a blood oxygen saturation level from the pulse-ox signal. The electronics generate an output signal that is based on the determined heartbeat rate and/or blood oxygen saturation level. An indicator proximate the electronics compartment is configured to receive the output signal and display an indication. A power source supplies electric power to the pulse oximeter sensor, electronics, and the indicator. The power source is recharged during use of the waterproof pulse oximeter sensor assembly by the swimmer.

### CROSS-REFERENCE TO RELATED APPLICATIONS

This application is a divisional of U.S. application Ser. No. 16/386,835, entitled “Flashing Indicator of a Swimmer's Health,” filed on Apr. 17, 2019, which is a continuation-in-part of U.S. application Ser. No. 14/755,020, issued as U.S. Pat. No. 10,413,225, entitled “Pulse Oximeter Sensor Assembly and Methods of Using Same,” filed on Jun. 22, 2015, the entirety of which is incorporated by reference herein. U.S. application Ser. No. 16/386,835 further claims the benefit of and priority to U.S. Provisional Application Ser. No. 62/658,651, entitled “Flashing Indicator of Swimmer's Health,” filed on Apr. 17, 2018, the entirety of which is incorporated by reference herein.

### RIGHTS OF THE GOVERNMENT

The invention described herein may be manufactured and used by or for the Government of the United States for all governmental purposes without the payment of any royalty.

### BACKGROUND OF THE INVENTION

Field of the Invention The present invention generally relates to the field of diagnostic instruments including wearable sensors, and more particularly to a waterproof pulse oximeter sensor assembly adapted to be positioned on a subject's hand or wrist and methods of using same.

### DESCRIPTION OF THE RELATED ART

The U.S. Department of Defense trains service men and women who enter into specialized career fields such as combat control, pararescue, and other special operations positions in a variety of aquatic conditioning skills. The aquatic skills received enable military personnel to conduct operations around the world in and around water settings. However, prior to mastering these skills, basic recruits/students, hereinafter referred to as swimmers, are exposed to strenuous water sessions where swimmers are required to participate in both above and sub-surface activities with and without breathing apparatuses. These activities are often accompanied, per design, by high stress and physically demanding exercises where a swimmer's heart rate is elevated and blood oxygen saturation is reduced. Current aquatic conditioning sessions maintain a safe swimmer-to-instructor ratio to help identify when swimmers are in need of assistance through verbal and/or visual observation of a swimmer's physical state (still moving). However, when training activities require challenging subsurface exercises, instructors could benefit from a device which could provide enhanced visual notification of a swimmer's physiological condition. Moreover, a device that monitors the well-being of a swimmer who is reaching their individual physiological thresholds can also assist in improving the safety of the training environment.

Pulse oximeter (or pulse-ox) assemblies include sensors that are configured to measure heart beat rate and/or the oxygen saturation of the blood, and are of particular importance in emergency medical situations as well as the monitoring of patients with respiratory or cardiac problems. Generally, pulse oximeters operate by directing light, such as in the red and/or infrared wavelength range, from one or more light emitting diodes (LEDs) toward the skin and blood vessels. In operation, the pulse-ox assembly emits light from the LED(s), and a photodiode collects the light reflected from the subject's body (reflectance pulse oximetry) or transmitted through the subject's body part (transmissive pulse oximetry). Light in the red wavelength range is absorbed at a different rate than the infrared light. Accordingly, the ratio of oxyhemoglobin and deoxyhemoglobin can be calculated from the respective amounts of reflected or transmitted light.

To reduce potential interference, contemporary pulse-ox sensors are generally configured to be worn on nonintrusive portions of the body such as wrists, fingers, or ear lobes. The measured heart rate and blood oxygen saturation of the subject is then reported on a local digital display or transmitted using wireless communication connections (e.g. bluetooth) to a remote display or system. This technology approach is practical for use in hospitals, home settings, and aviation environments; however, it does little to no assistance in aquatic settings. RF wireless communication is ineffective when submerged in water, which significantly attenuates the signal rendering the remote monitoring capability useless. Self-monitoring is not practical as swimmers will be engaged in other cognitive and physical activities and will typically push their limits to the point where they may become incapacitated. Therefore, a need exists for standoff monitoring that can be observed in both above and below water settings and provide a visual notification rather than the processing of digital information. Moreover, wearing sensors on one's fingertip or wrist by traditional means is generally cumbersome while swimming and may become detached through vigorous activities.

### SUMMARY OF THE INVENTION

Embodiments of the invention address the need in the art and provide a waterproof pulse oximeter sensor assembly configured to monitor vitals of a swimmer. The waterproof pulse oximeter sensor assembly includes a pulse oximeter sensor configured to evaluate conditions in a finger or a wrist of a swimmer and to generate a pulse-ox signal in response thereto. A waterproof electronics compartment includes electronics that are configured to electrically receive the pulse-ox signal from the pulse oximeter sensor. The electronics are further configured to determine a heartbeat rate and/or a blood oxygen saturation level from the pulse-ox signal and generate an output signal that is based on the determined heartbeat rate and/or blood oxygen saturation level. An indicator is proximate the waterproof electronics compartment and configured to receive the output signal from the electronics and display an indication to an observer. The waterproof pulse oximeter sensor may also include a power source operable to supply electric power to the pulse oximeter sensor, electronics of the electronics compartment, and the indicator. The power source may be recharged during use of the waterproof pulse oximeter sensor assembly by the swimmer.

Some embodiments of the invention allow for wireless charging through magnetic capacitive induction. Internal to the housing of the electronics enclosure is a coiled antenna that can be tuned to for the purpose of wireless charging. A common practice is a swimmer “rest” by holding onto a pool's edge, boat platform, inner-tube, etc. while receiving instructions or awaiting their designated time to conduct aquatic tasks. These embodiments may leverage this “resting” state by incorporating induction charging that can be used to extend the initial battery state of charge.

Some embodiments of the invention allow for digital readout of SpO2/HR for real-time interrogating.

Some embodiments of the invention allow for on-demand occlusion of the overt/covert notification through a mechanical filter lens over the LED cluster IRIS lens. Swimmers are often required to participate in water sessions that are during the night-time or in a light-controlled facility to hone the swimmers skills. These embodiments may be equipped with a retractable IRIS lens that can limit the amount of light-energy emitting from its LED cluster to include closing it completely. Alternately, in some other embodiments, the brightness of the LED cluster may be directly adjusted from dim to bright via an on board voltage control.

Some embodiments of the invention allow for interchangeable and removable physiological sensors through a quick-disconnect interface. A benefit of this modular design is that health sensors continue to evolve producing higher fidelity/accuracy measurements thereby supporting various sensors (both transmissive, reflective, etc.). These embodiments can scale and be customized to the environments and conditions required for military operations, for example. Moreover, having a dynamically adjustable pull-force quick-disconnect enables these embodiments to remain secure but not create a potential risk to the swimmer in case of an entanglement situation. The orientation of the quick-disconnect interface promotes custom cable lengths and promotes in-line cable routing minimizing drag impact to the swimmer.

Some embodiments of the invention possess the ability to harvest kinetic, thermal deltas, and induction tuning to supply energy to its control circuitry. Kinetically, these embodiments may leverage electro-magnetic energy generation as the wearer exerts continuous movement through various swimming maneuvers and strokes. Thermally, these embodiments may leverage an array of thermoelectric generators arranged on one side to rest on the skin of the wearer and the other side exposed to the water. Water temperatures and fluid dynamics provide a constant thermal delta enabling energy harvesting. Lastly, using magnetic induction, these embodiments may be able to be remotely powered through induction tuned energy waves created in the aquatic environment, thereby affording continuous monitoring without the risk of power depletion.

Some embodiments of the invention may leverage a chemical reactive fuel cell that can harvest power from the water surrounding the embodiments. Micro-perforated inlets allow an absorption membrane to gather and store fluids that when exposed to the fuel-cell an electrical reaction occurs producing current and voltage that are stored in the rechargeable battery cells of these embodiments.

Some embodiments of the invention allow for programmable thresholds of swimmers' vitals. These embodiments can be configured to capture a swimmer's baseline vitals and can be programmed to alert via the LED notification cluster when a swimmer is outside of his/her “safe” programmed operational vital range.

Some embodiments of the invention are able to archive a swimmers vital history, sampled at a predefined interval, to be offloaded at the completion of a swimming session. This feature allows swimmers to monitor their conditioning as well as provide instructors with information on a swimmer's state during key elements/exercise within training regimes.

Some embodiments of the invention may wirelessly transmit a swimmer's real-time health vitals using a configurable microcontroller emitter that senses the swimmer's environment, dynamically adjusting the method of transmission. These embodiments possess a variety of communication emitters to include but not limited to Radio Frequency (Bluetooth, Ultra-Wide Band, WiFi, Zig-bee), Magnetic Induction, and Optical Free-Space Communication. The communication microcontroller can sense the receipt of its transmission and determine if switching to another medium is required to output the swimmer's vitals.

Some embodiments of the invention can give instructors an on-demand indicator of the swimmers' readiness status following strenuous activities. These embodiments may contain an optical receiver that allows an instructor to remotely interrogate ON/OFF and/or activate the on-board digital readout display and/or powers-on the notification signal indicating the swimmers' readiness level remotely.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the invention and, together with a general description of the invention given above, and the detailed description given below, serve to explain the invention.

FIG. 1 is a view of a subject-facing side of a pulse-ox sensor assembly, in accordance with an embodiment of the present invention;

FIG. 2 is a side view of the pulse-ox sensor assembly shown in FIG. 1, in accordance with an embodiment of the present invention;

FIG. 3 is a view of an outward-facing side of the pulse-ox sensor assembly shown in FIG. 1, in accordance with an embodiment of the present invention;

FIG. 4 is top view of the pulse-ox sensor assembly shown in FIG. 1, in accordance with an embodiment of the present invention;

FIG. 5A is a schematic showing the location of a temporal artery in a human subject;

FIG. 5B is a schematic showing placement of the pulse-ox sensor assembly shown in FIG. 1 onto a subject's temporal region overlaying the temporal artery, in accordance with an embodiment of the present invention;

FIG. 6 is a perspective view of a pulse-ox sensor assembly, in accordance with another embodiment of the present invention;

FIG. 7 is a schematic showing placement of the pulse-ox sensor assembly shown in FIG. 6 onto a subject's temporal region, in accordance with another embodiment of the present invention;

FIGS. 8A-8C illustrate a hand/arm mounted arrangement consistent with an embodiment of the invention;

FIG. 9 is a diagrammatic representation of an instructor remotely interrogating the hand/arm mounted arrangement of FIGS. 8A-8C;

FIG. 10 is in isometric view of an electronic compartment of the hand/arm mounted arrangement of FIGS. 8A-8C;

FIG. 11 is a front view of the electronics compartment of FIG. 10;

FIG. 12 is a back view of the electronics compartment of FIG. 10;

FIG. 13 is a side view of the electronics compartment of FIG. 10;

FIG. 14 is a top view of the electronics compartment of FIG. 10; and

FIG. 15 is a bottom view of the electronics compartment of FIG. 10.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

Military personal who enter into specialized career fields such as combat control, pararescue, and other special operation forces and special tactics positions are required to master aquatic skills. Although at times, aquatic conditioning may prove to be too strenuous for swimmers and potentially necessitate medical intervention. The assemblies and methods described herein provide alerts when vital signs (e.g., SPO2 and/or HR) and core body temperature deteriorate to a predetermined threshold value prior to the need for medical intervention and thereby enable standoff monitoring of a swimmer's vitals and condition. Embodiments described herein provide an assembly and method for sub-surface (underwater) and/or aquatic (within) monitoring of various vital signs (e.g., heart rate (HR), blood oxygen saturation (SPO2), core temperature, etc.) of a subject in an aquatic environment and providing the subject and/or others with external overt notifications. However, it should be further appreciated that the assemblies described herein may also be utilized in non-aqueous environments.

Thus, in accordance with embodiments of the present invention, a health status sensor assembly is provided that includes 1) a pulse-ox sensor, 2) an indicator device, 3) a processor, 4) a base layer, 5) a mounting assembly, 6) a temperature sensor, and 7) a pressure sensor. The pulse-ox sensor is configured to facilitate measuring a subject's heart beat rate, blood oxygen saturation level, or both. The indicator device provides a discernible signal to the subject or others, (e.g., communication via optical modulation to an underwater and/or above water receiver). The processor, which is coupled to the pulse-ox sensor, temperature sensor, pressure sensor, and/or the indicator device, includes control logic configured to receive an output signal from any of the sensors, determine the subject's heart beat rate, blood oxygen saturation level, core body temperature, water temperature, atmospheric pressure, and generate an output signal for the indicator device that is based on the subject's heart beat rate, blood oxygen saturation level, and/or core body temperature. The base layer is configured to conform to a temporal region of the subject's head and includes one surface adapted to face and contact the subject's skin, and a second surface adapted to face away from the subject's skin. The mounting assembly is configured to enable securing and/or maintaining the health status sensor assembly over the temporal region of the subject's head, which, relative to more distal extremities (e.g., fingers or wrists), is less prone to blood pooling. The health status sensor assembly may also include a power supply, which can include a chamber that is adapted to hold and maintain a battery in electrical communication with the pulse-ox sensor, temperature sensor, pressure sensor, the processor, and the indicator device.

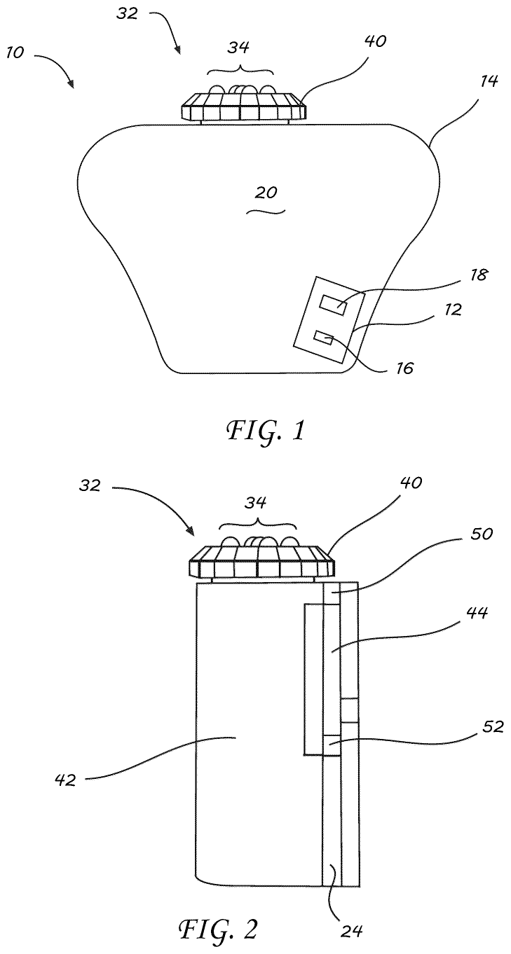

With more detail and specificity, the foregoing exemplary components are further described with reference to FIGS. 1-4, which provide complementary views of an exemplary embodiment. Referring to FIG. 1, an exemplary health status sensor assembly 10 is provided with a pulse-ox sensor 12 disposed in a base layer 14. The pulse-ox sensor 12 includes at least one light emitting diode (LED) 16 configured to emit light such as in the red and/or infrared range and at least one photodetector 18 configured to receive the light emitted by the at least one LED 16 and reflected by a subject's body (not shown) when in use and disposed proximate the body. The LED 16 and photodetector 18 cooperate to evaluate conditions observed in a region of the subject's skin, e.g., the temporal region. One exemplary pulse-ox sensor 12 is an 8000R Reflectance Sensor available from Nonin Medical, Inc. According to the depicted embodiment in FIG. 1, the pulse-ox sensor 12 is adapted to fit within an opening formed in the base layer 14. According to another embodiment, the pulse-ox sensor 12 may be embedded in the base layer 14. In either arrangement, it is advantageous that the pulse-ox sensor 12 does not protrude too far above a subject-facing side 20 of the assembly 10 as to cause undue discomfort to the subject. In one embodiment, the subject-facing surface 20 of base layer 14 and the pulse-ox sensor 12 are substantially uniplanar. In yet another embodiment, the pulse-ox sensor 12 is within about 0.5 mm, about 1 mm, about 2 mm, about 3 mm, or about 4 mm of the subject-facing surface 20 of the base layer 14. In yet another embodiment, base layer 14, could have a two-axis slidable adjustment for the pulse-ox sensor 12 to accommodate custom fitting to the subject's temporal artery.

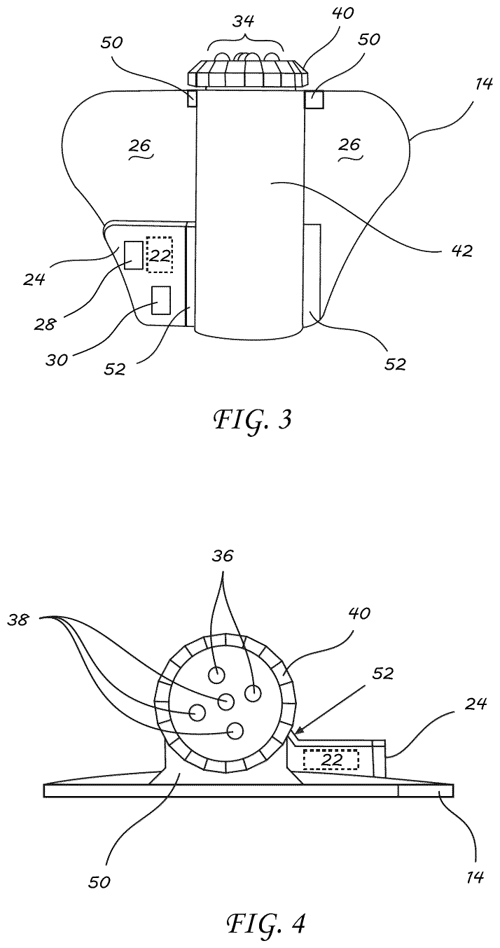

The pulse-ox sensor 12 is configured to electrically communicate with a processor 22, which may be contained in and protected by an electronics compartment 24. To permit continual use of the assembly 10 in a water environment, the electronic compartment 24 may provide a water-proof or water-resistant containment of the processor 22, as well as any other water-sensitive electronic components. The electronics compartment 24 is preferably made of waterproof material. In other embodiments of the invention, the electronics compartment 24 may be made of materials that are merely water resistant. As used herein, “water resistant” refers to material that is capable, at least, of resisting or impeding the passage of liquid water by mass flow, capillary or wicking action. Thus, water resistant material may not prevent all passage of liquid water under pressure. “Water tight” refers to connections between components that are at least water resistant. As used herein, “waterproof” refers to the capability of withstanding immersion in water. Such immersion could be as little as splashing or dropping the assembly on or in water, or by submersion to a depth of about 10 cm, about 1 m, about 10 m, about 38 m, or about 100 m, in sea or freshwater. Waterproof material can range from gas proof to gas permeable, including such breathable fabrics and fabric coatings such as Gore-Tex®. The electronics compartment 24 may in certain applications be constructed to be not only waterproof, but also gas-proof, such that gases cannot escape or enter the sealed electronics compartment 24. The assembly 10 may therefore be particularly useful to swimmers and divers. As shown in FIGS. 3 and 4, the electronics compartment may be positioned on an outward-facing surface 26 of the base layer 14.

The processor 22, which is coupled to the pulse-ox sensor 12, a temperature sensor 28, a pressure sensor 30, and an indicator device 32, may include control logic configured to a) receive an output signal from the pulse-ox sensor 12, b) determine the subject's heart beat rate, blood oxygen saturation level, or both, and c) generate an output signal for the indicator device 32 that is based on the subject's heart beat rate, blood oxygen saturation level, or both. The output signal may include data that provides numerical values of the measured vital signs, or simply designate if a threshold value for a vital sign has been crossed. The threshold value for a specific vital sign may be a value set within control logic of the processor 22 or it may be a value that is self-adjusting based on certain trials, exercises, routines, etc. For example, the threshold value may be based on one or more exercise routines (i.e., empirically derived), which can then be stored in memory, thereby permitting personalization of the threshold values for a given subject. The threshold value may also be based on multiple previous uses, which then updates the threshold value based on averaging the current use with the multiple previous uses.

In addition to the processor 22, other electronic components that may be present in the electronic compartment 24 include, but are not limited to, a network interface, a data storage device, and/or electrical connections therebetween. When present, the network interface can be configured to communicate data by a protocol such as universal serial bus (USB), Wi-Fi, Bluetooth, IrDA, radio frequency, IEEE 802.11, IEEE 802.15, Zigbee, and/or free space optical (FSO) communication. When present, the data storage device can include local, network-accessible, removable/nonremovable, volatile/nonvolatile, and/or transitory/nontransitory memory, such as RAM, ROM, firmware and/or flash and can be further configured to store program instructions that, when implemented by the processor, are configured to communicate with the pulse-ox sensor 12 and specifically to operate the LED(s) 16 and photodetector(s) 18 so as to receive data/information based on light reflected from the patient's body when the pulse-ox is in use. One or more of the processor 22, network interface, power supply, and data storage device can be configured to mount on a printed circuit board, each or all of which may be individually or collectively covered by waterproof or water-resistant materials. In some embodiments of the disclosed invention, substantially all electronic components may be encapsulated in an epoxy, urethane, silicone, resin, or the like, to seal against contamination ingress.

In an embodiment, the indicator device 32 may provide information relating to the absolute values of the measured vital signs. In another embodiment, the indicator device 32 may provide information as to the measured vital signs relative to the threshold value. In yet another embodiment, the indicator device 32 may provide information for both the absolute measurement and its relation to the threshold value. Accordingly, the indicator device 32 may provide the information visually, audibly, haptically, or a combination thereof, and may transmit the information for standoff monitoring. For example, the indicator device may include a light source, such as constant or flashing lights, one or more LEDs, or LCD displayed messages; a noise source, such as speakers that provide an audible tone; or a haptic device in contact with the subject that emits vibrating pulses; and/or a radiation transmitter. By way of further example, the indicator device 32 may be adapted to provide audible beeps through bone conductive transducers and tactile vibration along with visual alerts. Further, standoff notification alternatives can use a variety of frequencies to be picked up underwater and rebroadcasted through other protocols in pool settings. Moreover, each health status sensor assembly 10 may have a unique frequency output, or a common frequency with uniquely coded device identifiers, allowing for specific notification for individual swimmers.

In accordance with the embodiment shown in FIGS. 1-4, the indicator device 32 may provide a visual notification through an LED array 34, which may be a colored, multi-spectral LED cluster and may alert other subjects or standoff observers when the subject requires medical intervention, crosses a vital sign threshold, or is within a safe vital sign range. For example, the LED array 34 may comprise at least a first color indicator LED 36 and a second color indicator LED 38. Additionally, the LED array, the output of which may be modulated, may provide a positive identification for swimmers above surface and sub-surface. Furthermore, the modulation of the LED array 34 may provide the means for free space optical (FSO) communication to off-board receivers transmitting the swimmers state. In accordance with an embodiment, the LED array 34 may be conveniently housed in a cap 40 of battery chamber 42.

It will be readily apparent that any convenient portable power source (i.e., a battery) can be adapted to supply the electrical requirements for the health status sensor assembly 10. In an embodiment, the battery is contained within a battery chamber 42. To enable easy battery replacement, the battery chamber cap 40 and the battery chamber 42 may form a resealable, watertight connection (e.g., a screw cap with rubber O-ring). It should be appreciated that the form of the battery chamber 42 may modified or customized to accommodate virtually any battery shape. For example, as shown in FIG. 2, the battery chamber 42 is formed to accommodate a cylindrical battery, such as a typical AA or AAA battery.

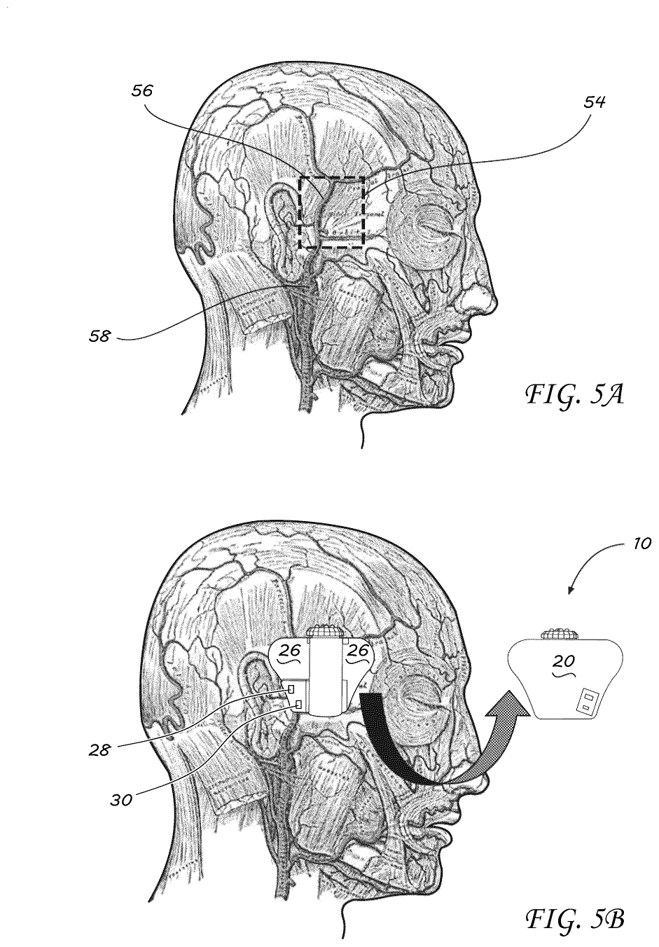

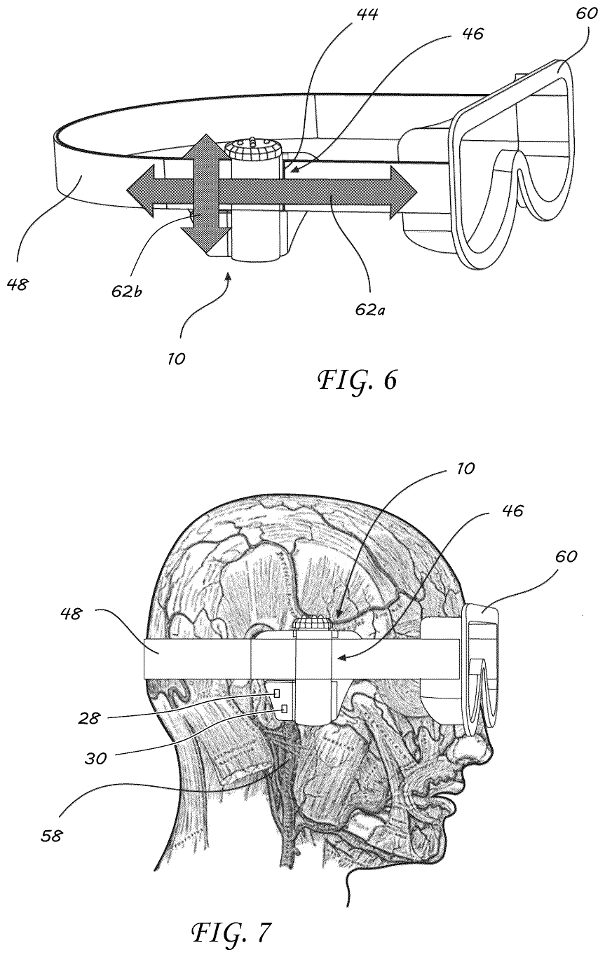

In accordance with another embodiment, the battery chamber 42 may be connected or joined to the base layer 14 in a manner that provides an aperture 44 between the two, which may serve as a mounting assembly 46 to accommodate a retaining strap (e.g., a goggle strap 48, as shown in FIGS. 6 and 7). For example, the battery chamber 42 may be connected to the base layer 14 by an upper support 50 and a lower support 52, which thereby forms the aperture 44. For the cylindrical battery chamber 42 shown in FIGS. 2-4, the aperture-facing portion of the battery chamber 42 may be modified to convert the curved surface to a generally planar surface that is approximately parallel to the outward-facing surface 26 of the base layer 14. Accordingly, the aperture 44 may be configured to accommodate or engage with a head band, a goggle strap, or an eye glass temple to hold and maintain the position of the pulse-ox sensor 12 over the temporal region 54, and more specifically the temporal artery 56 (see FIGS. 5A and 5B). The temporal artery 56 is the smaller of two terminal branches (the other is the maxillary artery) that bifurcate superiorly from the external carotid artery 58 (see FIG. 5A).

The base layer 14, which includes the pulse-ox sensor 12 on the subject-facing surface 20, helps to secure the assembly 10 flush to the subjects' temporal region and prevents the assembly 10 from rotating or being displaced during exercise (e.g., swimming) activities. In accordance with an embodiment, the base layer 14 is configured with a large surface area flare design, relative to the surface area of the pulse-ox sensor 12. For example, a typical pulse-ox sensor 12 may have a surface area that is about 0.3 cm² to about 0.5 cm², and the base layer 14 may have a subject-facing surface area that is about 5×, about 6×, about 7×, about 8×, about 9×, about 10×, or more.

Additionally, the base layer 14 is configured to conform to a temporal region 54 of the subject's head. For example, the base layer 14 may be generally isosceles trapezoidal or “butterfly” in shape The location of the pulse-ox sensor 12 within the two-dimensional area of the subject-facing surface 20 of the base layer 14 need not be fixed to one specific area. However, placement of the pulse-ox sensor 12 centrally within the relatively larger surface area of the base layer 14 may require more adaptation to accommodate the ear tissue of the subject. While not shown, a blunted protuberance (e.g., an ear bud) may be projected from the subject-facing surface to facilitate indexing the pulse-ox sensor 12 over the temporal artery 56. In conjunction with the foregoing, the ear bud may also provide a conduit for transmitting an audible signal from the processor to the subject.

The base layer 14 and other components that contact the subject's tissue (i.e., skin and/or hair) may be made of any suitable material, such as biocompatible materials. Flexible polymeric materials, such as polyethylene, polypropylene, fluoropolymers (e.g. polytetrafluoroethylene), polyurethane, or other similarly water resistant or waterproof materials, may be suitable for use. While latex materials are not specifically excluded, the use of latex may be avoided to prevent adverse reactions induced by latex allergies.

The aperture 44 may be configured to accommodate or engage with a head band, a goggle strap, or an eye glass temple to hold and maintain the position of the pulse-ox sensor 12 over the temporal region 54. In addition to embodiments shown in FIGS. 2-4, where the aperture-facing portion of the battery chamber 42 is shown modified to convert the curved surface to a generally planar surface that is approximately parallel to the base layer 14, other configurations or devices may be use in the mounting assembly 46. In an alternative embodiment (not shown), a clip or loop may be fixed to the outer-facing surface of the battery chamber 42. In yet another embodiment (not shown), one or more clips or loops may be fixed to the outer-facing surface 26 of the base layer 14 that are laterally disposed from the battery chamber 42. In any of the foregoing alternative embodiments, the clip(s) or loop(s) may be configured to accommodate or engage with a head band, a goggle strap, or an eye glass temple to hold and maintain the position of the pulse-ox sensor 12 over the temporal region 54, and more specifically the temporal artery 56 (see FIGS. 5A and 5B). It should be further appreciated that other structures or materials may be used to connect the assembly 10 to a head band, a goggle strap, or an eye glass temple, such as hook and loop fasteners, snaps, clasps, stitching, or the like. In other words, the assembly 10 may be directly or indirectly connected to the head band, the goggle strap, or the eye glass temple.

As shown in FIGS. 6 and 7, in accordance with another embodiment, the mounting assembly 46 may further include a set of goggles 60 and a goggle strap 48. The goggle strap 48 passes through the aperture 44 formed between the battery chamber 42 and the base layer 14. Once the subject dons the combined pulse-ox sensor assembly 10, goggles 60, and goggle strap 48, the assembly 10 may be repositioned (as indicated by the bi-directional arrows 62a, 62b) to place the pulse-ox sensor 12 over the temporal artery 56. Due to the frictional force between the goggle strap 48 and internal walls of the aperture 44, as well as the stability provided to the assembly 10 by the relatively large surface area of the subject-facing surface 20, the assembly 10 resists displacement during vigorous exercise, such as swimming.

The health status sensor assembly 10 may further include a pressure sensor (not shown), which is also in communication with the processor. In aqueous environments, the pressure sensor may provide an output signal to the processor indicating whether the subject's head is underwater, and optionally a depth of water above the subject's head. Accordingly, the processor may further include control logic configured to receive the output signal from the pressure sensor, determine a position of the subject's head in relation to a water surface as the depth from the water surface is directly proportional to the change in pressure. Then, a second output signal may be generated for the indicator device that is based on the position of the subject's head in relation to a water surface. For example, different colored lights in the LED array 34 of the indicator device 32 may be used to identify whether the subject's head is submerged. Additionally, the output of LED array 34 may be modulated to differentiate when the subject's head is above or below the water surface. Moreover the pressure sensor may be used to toggle predefined/customized power saving modes. For example, the LED array 34 and/or the pulse-ox sensor 12 may only be operational and powered once the subject is below the water surface.

In non-aqueous settings, the pressure sensor may be used as a component of an altimeter to provide information regarding absolute or changes in elevation. Additionally, global positioning system (GPS) receiver may also provide information about its location and elevation above sea level.

In accordance with an embodiment of the present invention, one purpose of the assembly 10 is to provide monitoring and assessing the physical state of active personnel. The assembly 10 described herein enables standoff monitoring of a subject's vital signs (e.g., SPO2 and HR) and provides visual alerts to observers monitoring the subject when vitals deteriorate to a predetermined threshold point prior to the need for medical intervention. For example, the assembly 10 enables determining if the subject's heart beat rate is greater than the predetermined threshold heart beat rate, determining if the subject's blood oxygen saturation level is less than the predetermined threshold blood oxygen saturation level, or both.

In aqueous environments, the assembly 10 is a waterproof device that monitors an subject's key physiological vitals, (e.g., HR and SPO2 levels), and emits a visual notification, (e.g., a continuous light, a flashing light, illuminating different colors, etc.), when one or more vital signs reach predefined thresholds to alert observers of a warning state that the subject may be experiencing. The assembly 10 can provide an earlier positive identification when the monitored subject needs assistance, when a subject needs requires additional recovery time from exercise, and enables standoff monitoring. Advantageously, notifications to standoff observers are passively invoked requiring no interaction from the subject swimmer to initiate.

Accordingly, another embodiment of the invention includes a method for monitoring and assessing a physical state of a subject is provided. The method includes positioning the pulse-ox sensor assembly 10 over the temporal region of the subject's head, wherein the pulse-ox sensor 12 is positioned over a portion of the temporal artery; obtaining the subject's heart beat rate, blood oxygen saturation level, or both; comparing the subject's heart beat rate to a predetermined threshold heart beat rate, and/or comparing the subject's blood oxygen saturation level to a predetermined threshold blood oxygen saturation level; and transmitting a signal to the indicator device 32 of the assembly 10 that indicates if the predetermined threshold heart beat rate and/or the predetermined threshold blood oxygen saturation level have been crossed. As noted above, the method and the assembly 10 described herein enables standoff monitoring of a subject's vital signs (e.g., SPO2 and HR) and provides visual alerts to observers monitoring the subject when vitals deteriorate to a predetermined threshold point prior to the need for medical intervention. Alternatively, audible alarms, vibrational devices, and/or radiation transmitters may also provide notification of a warning state to the monitored subject. Advantageously, notifications to standoff observers are passively invoked requiring no interaction from the monitored subject to initiate.

While the above embodiments of the assembly 10 significantly improve the current state-of-the-art for aquatic heart rate (HR) and blood-oxygen (SPO2) monitoring, continual design and refinement in form, fit, function identified further enhancements that are proving novel and are beneficial. The embodiments of the assembly 10 set out above actively measure the swimmer's blood profusion of the temporal artery and triggers an overt LED notification signal once a swimmer's vital levels reaches a predefined level. While this notification is useful for many applications, there is an additional need for enhanced monitoring and remote triggering of a swimmer-worn SPO2 monitor that provides real-time health data to remote observers.

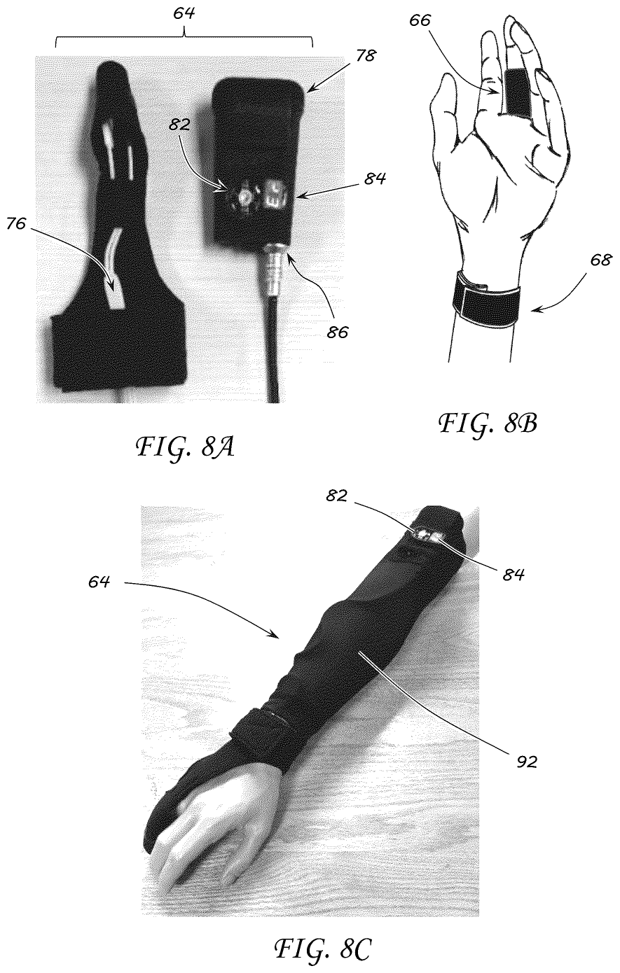

An alternate embodiment of the invention may be positioned on a finger 66 or a wrist 68 of a swimmer using a hand/arm assembly 64 illustrated in FIGS. 8A-8C. This hand/arm assembly 64 assists in providing a sub-surface aquatic (underwater) monitoring of heart rate and blood oxygen saturation of a swimmer with external overt notifications. This embodiment may also assist personnel who enter into specialized career fields such as combat control, pararescue, and other special operation forces and special tactics positions are required to master aquatic skills. At times, aquatic conditioning may prove to be too strenuous for swimmers and potentially result in the need for medical intervention. The hand/arm assembly 64 is a waterproof device that monitors an individual's key physiological vitals-heart rate, SPO2 levels, core body temperature—and emits a visual notification, flashing light, when vitals reach predefined thresholds to alert observers of a warning state a swimmer may be experiencing. These embodiments may also tie into pre-existing sensors to measure blood pressure through the use of a blood pressure “cuff” in a watch or arm band form factor and potentially measure blood glucose in vivo through the use of constant blood glucose sensors, among other types of vitals sensors. The hand/arm assembly 64 provides an earlier positive identification when swimmers need assistance or require additional recovery time from exercise, and provides standoff monitoring. Notifications are passively invoked requiring no interaction from the swimmer to initiate. Programmable thresholds of swimmers' vitals may be configured to capture a swimmer's baseline vitals. Then, additional levels may be programmed to alert via an LED cluster, for example, when a swimmer is outside of his/her “safe” programmed operational vital range.

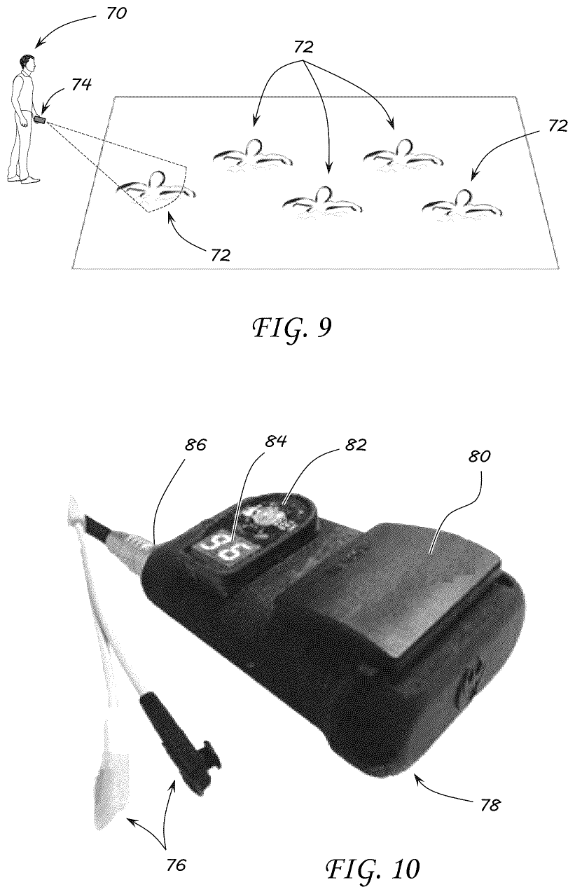

As illustrated in FIG. 9 the hand/arm assembly 64 provides instructors 70 an on-demand indicator of the swimmers' 72 readiness status following strenuous activities. The hand/arm assembly 64 may contain an optical receiver 74 that may allow an instructor to remotely interrogate ON/OFF and/or activate an on-board digital readout display and/or powers-on the notification signal indicating the swimmers' 72 readiness level remotely.

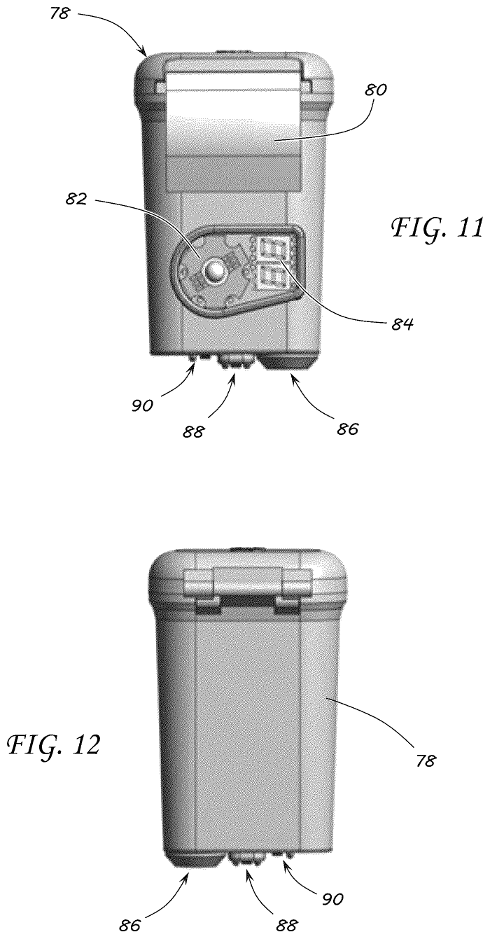

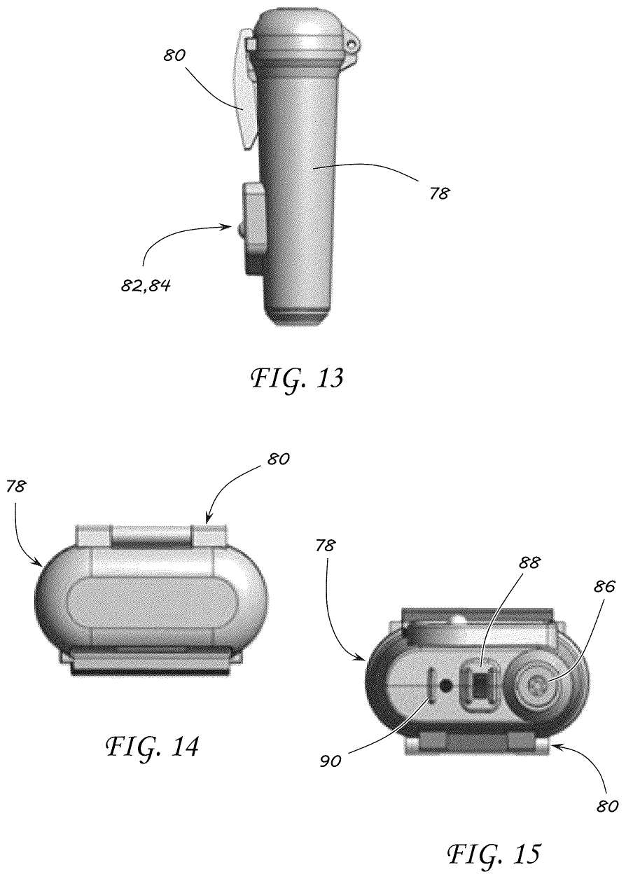

A specific embodiment of the hand/arm assembly 64 may include eight overarching elements as illustrated in FIGS. 10-15. These include a vital transmissive sensor 76, an electronic compartment with USB port or the like for data downloads 78, a securing clip 80, a LED alert cluster 82, a dual alphanumeric display 84, a water-proof LEMO connector 86, a power switch 88, and a mode switch 90.

Transmittance sensor 76 measures both oxygenated and deoxygenated hemoglobin through the use of red and infrared LEDs, similar to sensor 12 in assembly 10, and electrically communicates those measurements to electronics in the electronic compartment 78. The electronic compartment 78 may include digital microcontrollers, memory, communication drivers, and input/output electronics/protocols to process the data feed of the transmittance sensor 76, to drive the LED cluster 82, and to communicate with off-board systems similar to that of assembly 10 set out above, among other functions. Electronics in the electronic compartment 76 may also manage threshold levels, power, and may send control signals to the LED cluster 82 illuminating a series of LEDs, per the control signals, if a threshold is reached. The LED cluster 82 may contain a series of multi-colored visible and infrared LEDs for overt and covert visual notification in some embodiments.

The securing clip 80 secures a lid to the electronic compartment 78 against a rubber gasket maintaining waterproofing. LEMO Connector 86 is a water-proof connector that facilitates transmission of the signal from the transmittance sensor 76 to the electronics in the electronic compartment 78. The dual alphanumeric display 84 may display the SpO2 data output received by the electronics in the electronics compartment 78 as well as other values or message codes. For example, the display may, alone or in conjunction with the LED alert cluster, be utilized indicating to the medical responder that a patient is in need of assistance. In some embodiments assembly 64 may include a waterproof speaker in addition to the LED cluster 82 for alerting a medical responder. An auditory alert can accompany the visual alert.

Some embodiments of assembly 64 may allow for on-demand occlusion of the overt/covert notification through a mechanical filter IRIS lens over the LED cluster 82. Swimmers 72 are often required to participate in water sessions that are during the night-time or in a light-controlled facility to hone the swimmers skills. Some embodiments of assembly 64 may be equipped with a retractable IRIS lens that can limit the amount of light-energy emitting from its LED cluster to include closing it completely.

Assembly 64, similar to assembly 10, may also be battery powered. However, some embodiments of assembly 64 allow wireless charging through magnetic capacitive induction. Internal to the electronic compartment 78 is a coiled antenna that may be tuned for the purpose of wireless charging. A common practice is a swimmer 72 “rest” by holding onto a pool's edge, boat platform, inner-tube, etc. while receiving instructions or awaiting their designated time to conduct aquatic tasks. Assembly 64 leverages this “resting” state by incorporating induction charging that can extend the initial battery state of charge.

Additionally in some embodiments, assembly 64 may possess the ability to harvest kinetic, thermal deltas, and induction tuning to supply energy to its control circuitry or charge batteries. Kinetically, these embodiments may leverage electro-magnetic energy generation as the wearer exerts continuous movement through various swimming maneuvers and strokes. Thermally, these embodiments may leverage an array of thermoelectric generators arranged on one side to rest on the skin of the wearer and the other side exposed to the water. Water temperatures and fluid dynamics provide a constant thermal delta enabling energy harvesting. Lastly, using magnetic induction, these embodiments may be able to be remotely powered through induction tuned energy waves created in the aquatic environment, thereby affording continuous monitoring without the risk of power depletion.

Alternately, in some embodiments, assembly 64 may leverage a chemical reactive fuel cell that can harvest power from the water surrounding the assembly 64. Micro-perforated inlets allow an absorption membrane to gather and store fluids that when exposed to the fuel-cell cause an electrical reaction producing current and voltage that may stored in rechargeable battery cells and used to power the device.

In an exemplary embodiment, the vital transmittance sensor 76 may be securely mounted to the base of the finger 66, or the wrist 68, with an attached data module and alert system. This may be accomplished, in some embodiment, with a sleeve 92 as illustrated in FIG. 8C. Embodiments of assembly 64 provide for interchangeable and removable physiological sensors through a quick-disconnect interface. A benefit of this modular design is that health sensors continue to evolve producing higher fidelity/accuracy measurements thereby supporting various sensors (both transmissive, reflective, etc.). Embodiments may scale and be customized to the environments and conditions required for different sets of operations. Moreover, having a dynamically adjustable pull-force quick-disconnect enables these embodiments to remain secure but not create a potential risk to the swimmer in case of an entanglement situation. The orientation of the quick-disconnect interface promotes custom cable lengths and promotes an in-line design minimizing drag impact to the swimmer 72 wearing the assembly 64.

Assembly 64 allows for digital readout of SpO2/HR/Core Temperature for real-time similar to assembly 10 above. In some embodiments, electronics in the electronic compartment 78 may be configured to archive a swimmers 72 vital history, sampled at a predefined interval, to be offloaded at the completion of a swimming session. This feature allows swimmers 72 to monitor their conditioning as well as provide instructors with information on a swimmer's 72 state during key elements/exercise within training regimes.

Embodiments of assembly 64 may also wirelessly transmit a swimmer's 72 real-time health vitals using a configurable microcontroller emitter that senses the swimmer's 72 environment, dynamically adjusting the method of transmission. These embodiments may possesses a variety of communication emitters to include but not limited to Radio Frequency (Bluetooth, Ultra-Wide Band, WiFi, Zig-bee), Magnetic Induction, and Optical Free-Space Communication. The communication microcontroller can sense the receipt of its transmission and determine if switching to another medium is required to output the swimmer's 72 vitals.

Finally, power switch 88 turns the system on and off and mode switch 90 cycles the system through various sensor modes.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

What is claimed is:

1. A health status sensor assembly configured to monitor vitals of a swimmer, the health status sensor assembly comprising:
a waterproof housing configured to be positioned proximate to a temporal artery of the swimmer;
a temperature sensor operably coupled to the waterproof housing and configured to detect a water temperature, the temperature sensor being further configured to provide a temperature sensor output signal;
a pulse oximeter sensor operably coupled to the waterproof housing and configured to evaluate conditions in the temporal artery of the swimmer and to generate a pulse-ox signal in response thereto;
a pressure sensor operably coupled to the processor and configured to detect a change in pressure, the pressure sensor being further configured to provide a pressure sensor output signal; and
a processor in the waterproof housing and operably coupled to the pulse oximeter sensor, the temperature sensor, and the pressure sensor, wherein the processor includes control logic configured to:
receive the pulse-ox signal from the pulse oximeter sensor, the temperature sensor output signal from the temperature sensor, and the pressure sensor output signal from the pressure sensor,
determine a heartbeat rate, a blood oxygen saturation level, a core body temperature, or combinations thereof from the pulse-ox signal,
determine an underwater depth in relation to the detected change in pressure,
generate an output signal that is based on the determined heartbeat rate, blood oxygen saturation level, the core body temperature, the underwater depth, the water temperature or a combination thereof, and
transmit the output signal to an indicator

2. The health status sensor assembly of claim 1, further comprising:
an indicator operably coupled to the waterproof housing and configured to receive the output signal from the processor and, in response to the output signal is configured to transmit a visual signal, an audible signal, a sensory signal, or a combination thereof.

3. The health status sensor assembly of claim 2, wherein the indicator is further configured to transmit the output signal at a set frequency for standoff monitoring.

4. The health status sensor assembly of claim 2, wherein the indicator comprises a light source, a noise source, a haptic device, or combinations thereof.

5. The health status sensor assembly of claim 1, further comprising:
a network interface in the waterproof housing configured to communicate data from the processor by a protocol selected from USB, Wi-Fi, Bluetooth, IrDA, radio frequency, IEEE 802.11, IEEE 802.15, Zigbee, Free Space Optical, or combinations thereof.
